(19) United States
(12) Patent Application Publication
(10) Pub. No.: US 2026/0284622 A1
(43) Pub. Date: Sep. 24, 2026

(54) TUBE BUNDLE REACTOR

(71) Applicant: MAN ENERGY SOLUTIONS SE, Augsburg (DE)

(72) Inventors: Klaus EHMANN, Deggendorf (DE); Andreas GOLDMANN, Eggenfelden (DE); Rainer NOWAK, Metten (DE)

(21) Appl. No.: 19/149,958

(22) PCT Filed: Jan. 11, 2024
(86) PCT No.: PCT/EP2024/050611

§ 371 Date: Jul. 22, 2025

(30) Foreign Application Priority Data
Jan. 26, 2023 (DE) 10 2023 101 925.2

(51) Int. Cl. B01J 8/06; B01J 8/00; C07C 45/28; C07D 307/60; C07D 307/89

(52) U.S. Cl. CPC B01J 8/065; B01J 8/001; C07C 45/28; C07D 307/60; C07D 307/89; B01J 2208/00539; B01J 2208/021

(57) ABSTRACT

A tube bundle reactor for performing catalytic gas phase reactions, having a bundle of catalyst-filled reaction tubes, a gas-inlet-side tube base, a gas inlet hood, which spans the gas-inlet-side tube base, and having a throttle plate, which is arranged upstream of the gas-inlet-side tube base and thus forms an intermediate space between the throttle plate and the gas-inlet-side tube base and has a predetermined number of throttle openings, the throttle openings have an extremely narrow flow cross-section and the throttle openings are arranged in such a number as to result in a sub-critical pressure ratio at the throttle plate in normal operation, and in a critical pressure ratio at the throttle plate in the event of an explosion. As a result of the gas inflow from the throttle plate and the gas outflow to the reaction tubes a predetermined pressure in the intermediate space is not exceeded.

### CROSS REFERENCE TO RELATED APPLICATIONS

This is a U.S. national stage of Application No. PCT/EP2024/050611 filed Jan. 11, 2024. Priority is claimed on German Application No. DE 10 2023 101 925.2 filed Jan. 26, 2023, the contents of which are incorporated herein by reference.

### BACKGROUND OF THE INVENTION

### 1. Field of the Invention

The present disclosure relates to a tube bundle reactor and to a method for operating and using such a tube bundle reactor.

### 2. Description of the Related Art

A generic tube bundle reactor is known from EP 3 700 663 A1. It comprises a mixing chamber for forming gas mixtures which, at least at times, can be present in an ignitable state without undesirable ignitions and subsequent explosions or deflagrations or detonations occurring. In the process, a first gas is fed through a partition wall provided with passages in a critical state of flow into a mixing chamber, in which distributors for a second gas are located. A critical state of flow is present in particular when a compressible fluid in the narrowest flow cross-section of a throttle opening reaches the speed of sound. In this state of flow, the mass flow is only dependent on the upstream pressure. Relevant technical literature in this regard is stated, for example, in the abovementioned EP 3 700 663 B1. A critical state of flow can be adjusted in particular through a suitable selection of number and diameters of the throttle openings with given upstream pressure.

By mixing the process gas in EP 3 700 663 B1 just shortly before entering the reaction tubes, only a part of the reactor has to be designed so as to be explosion-proof. The passages in the limit wall are preferably embodied as nozzles with Venturi or Laval characteristic. Preferably, separate nozzles configured in a defined manner are used, which are, for example, screwed into the limit wall. Furthermore, particles, packings or static mixers with or without catalytic action can be introduced into the mixing chamber. The mixing chamber is preferably of explosion-proof design and the limit walls are braced against one another with stay bolts so as to reduce the wall thicknesses of the limit walls.

The permanent flow through the limit wall in the critical state of flow results to a correspondingly high loss of pressure, which results in substantial costs in the equipment and during the operation. This applies all the more when the two process gas components are of the same order of magnitude. According to the system, the mixing time of the reaction gas components in such designs is very short and accordingly the mixing quality low, which can result in reaction conditions that are not defined. In addition, the devices require a major design effort.

Generic tube bundle reactors are based on the consideration of achieving as high as possible a production performance relative to the size of the reactor plant, in that the charging of the process gas with the explosion-critical components such as for example, oxygen and hydrocarbon is made as large as possible. However, by doing so one ever further enters the explosive concentration range and the probability of an explosion increases.

During regular operation, process gases can have an explosive composition or they are not explosive but it cannot be ruled out in the event of a fault that explosive gas compositions form. In any case, explosions can occur in the gas inlet region of a tube bundle reactor despite all precautionary measures. Explosions can have different characteristics. When the pressure wave of a combusting gas propagates at subsonic speed this is referred to as a deflagration. When the pressure wave reaches supersonic speed, this is referred to as detonation. The pressure developing in the process is far greater than the pressure caused by the deflagration. In certain conditions, a deflagration can merge into a detonation. In the cases discussed here deflagrations will generally form.

From the prior art, different structures for protecting different regions of a gas space subjected to an explosion pressure are known. Pressure waves as a consequence of the explosion of explosive gases can occur in open systems at atmospheric conditions or in closed systems with static or flowing medium under atmospheric conditions or under pressure. For damping the effects of such pressure waves, numerous devices are known.

For protecting a tube bundle reactor and connected plant components from damage or even destruction as a consequence of such pressure shocks, there are different measures depending on the application case. As a first measure, attempts are basically made to exclude sources of ignition. When explosive gas compositions are present and explosions cannot be ruled out, further measures are necessary. The explosion pressure can be diminished through explosion relief. This mostly involves loud pressure waves. When in the process dangerous or toxic substances additionally escape or corresponding pressure relief devices for given response pressures are not available, this route is not applicable. In addition, the plant has to be taken out of service in order to replace the mostly destroyed pressure relief devices. A further possibility consists in embodying the apparatus components subjected to explosion pressure in a pressure-encapsulated manner, i.e. the apparatus is embodied so sturdy in terms of strength that it withstands an explosion pressure.

When a pressure wave is closely related to the causal flame front, the flame front can be effectively dampened through heat-extracting means during the passage of the flame front or be entirely brought to a halt. When the damping of the pressure wave alone is of primary importance, porous media of very distinct types were developed for this purpose. For example, different fabrics, meshes, porous monolithic bodies or perforated plates with subcritical or supercritical flow, other installations and combinations thereof are known.

Thus, flow obstacles which absorb the pressure or heat energy in different ways are always arranged. All known devices have in common that these are the more effective the smaller the flow passage or the porosity is. The absorbed energy has to be absorbed either directly or be diverted to the wall of a flow channel.

A tube bundle reactor known from WO 2004/067164 A1 contains a gas inlet space, which is formed out of the volume of the gas inlet hood, the adjoining gas inlet line and a part of a non-return valve arrangement, wherein the valve plate of the same, with non-return valves arranged thereon, forms the upstream limit of the gas inlet space. In terms of strength, all limits of this gas inlet space are designed for the maximum pressure that occurs during the explosion or detonation of the process gas. This is done through suitable wall thicknesses and support structures on the gas side and also on the side of the heat transfer medium in the region of the tube bundle. Furthermore, the probability of explosion occurrences is lowered by minimising volumes in the region of the gas inlet hood with poor flow.

For intercepting a pressure shock resulting from an explosion for protecting upstream plant components an evaporator having a perforated screen basket is additionally known from DE 10 2004 052 827 A1, which is used here primarily before a dosing apparatus of liquid substances.

DE 20 2005 012 725 U1 relates to a similar evaporator. The mixing of reaction gas components is improved through a cross-sectional constriction and any explosion pressure shocks are dampened by multiple screen baskets.

According to DE 198 06 810 A1, the temperature of the gas-inlet-side tube base can be lowered by a heat insulation layer applied to it in order to prevent harmful side reactions including ignitions and deflagrations.

In a similar manner, WO 2000/51962 A1 uses a filter and distribution bed of inert material for filtering out and evenly distributing liquid constituent parts over the reaction tubes.

In the case of WO 2005/016509 A1, a short bed of packing material is arranged before the inlets of the reaction tubes of a tube bundle reactor, which discharges contaminations from heat exchange media, thus preventing the formation of combustible gas mixtures. EP 2 106 848 A2 describes a method for mitigating a deflagration pressure, wherein at least 20% of a gas space adjoining a tube base is filled with a particulate mitigation material. The gas inlet space can also be completely filled with the mitigation material. The method includes the experimental determination of a function which describes the dependency of the peak deflagration pressure on the mean particle diameter. On the basis of this determined dependency, a mitigation material with a smaller particle diameter than the particle diameter of a permissible deflagration pressure is used in the tube bundle reactor.

From EP 3 381 519 A1 and EP 3 381 520 A1 a method or a system is additionally known for testing a combustion barrier, wherein the combustion barrier to be tested is located between two gas spaces with static gas.

A further function principle for damping or extinguishing explosion pressure waves consists in extracting heat energy from the flame front, thereby causing the flame to be extinguished. Such a flame barrier is known from DE 2 225 552 A1. The publication relates to a safety device for installation in a line conveying ignitable gas or gas mixture for preventing the wave propagation of re-ignitions, flame strikes, deflagrations and the like. The safety device includes a housing with a flow element of a multiplicity of parallel plates with subdivisions, which subdivide the intermediate spaces into channels with cross-sectional dimensions of approximately 1 mm.

The publication WO 2000/56406 A1 relates to a similar detonation flame barrier, wherein the flame barrier of the flow element is subdivided into two thinner spaced-apart elements.

In the case of WO 1994/000197 A1 the flow element consists of multiple disc-shaped elements each with a multiplicity of axially parallel flow channels, wherein swirl generators are situated between these elements.

The publication EP 0 568 326 A1 discloses a detonation barrier having multiple flow elements parallel in the axial direction, which have elevated heat dissipation capacities and thus a longer resistance capability. Apart from this, central baffle plates with smaller diameters than the flow elements are arranged before the flow elements in order to mitigate the centre of the pressure wave.

Further structures for mitigating the centre of the pressure wave through baffle plates or deflection elements are known, for example, from WO 2021/253828 A1, DE 195 36 292 A1 and DE 298 23 686 U1.

The flow elements according to DE 24 36 206 A1 can be protected against the force of an explosion pressure wave by reinforcing elements.

The effectiveness of flame and detonation barriers can be amplified by connecting multiple circular plate-like flow elements in series, such as for example, is shown in DE 44 38 797 C1, WO 2001/37933 A2, WO 2001/41876 A2, KR 101363444 B1 or in WO 2018/078814 A1.

For quenching a flame front, a tube bundle cooled from the shell-side is also known from WO 2016/081559 A1.

The flow element of a flame barrier in EP 1 044 703 A2 consists of a multiplicity of parallel plate strips arranged into a stack, which are spaced apart from one another by protrusions. In the flame barrier according to WO 2018/053331 A1 the flow element is formed of multiple layers of wire mesh. In the case of US 2018/0056100 A1 and US 2018/0056101 the flow element consists of monolithic bodies which are traversed by straight or curved channels of different shape. Similarly, CN110575635 A shows a flame barrier, which consists of at least two parallel and spaced-apart plates produced by 3D-print, wherein the plates are traversed by serpentine flow channels.

From US 2004/0074537 A1 a device for limiting the tapping air flow from an engine of an aircraft for use in the sub-systems of the aircraft. For limiting the energy expenditure, such devices are to preferably have a low pressure loss. A one-piece Venturi device typically used in such applications with a converging section, a neck and a diverging section and correspondingly great length is replaced by a device of identical cross-section with a multiplicity of smaller parallel Venturi nozzles with smaller flow cross-sections and correspondingly significantly shorter length. Each flow path includes a Venturi device with a neck and an associated diverging section preferably also with a converging section.

DE 24 43 923 A1 describes a device with a single throttle for installation in a pipeline, wherein the narrowest flow cross-section of a compressible flow (“vena contracta”) coincides with the geometrically narrowest flow cross-section. Thus, uncertainties in the calculation of the pressure loss are reduced. The cone angle before the narrowest flow cross-section is in a range between 4° and 40°. Further, conical sections can be arranged before and after this functional cone. The throttle can operate as a device for flow limitation, in that during normal operation it is operated in the critical state of flow and thus, only permits a certain maximum flow rate. Thus, any breakage of the line downstream of the throttle does not cause any enlargement of the flow rate.

U.S. Pat. No. 5,413,145A describes a Venturi nozzle for normal operation in the critical state of flow. Through mirror-image configuration of inlet and outlet with circular contour and without cylindrical transition, the manufacture is cost-effective. By way of the directional independence the installation is simplified.

An expansion system according to DE 10 2012 014 100 A1 serves in an emergency for the quick or emergency expansion of a pressurised pressure system and generally comprises a flare system operated without pressure. In an emergency, the expansion system causes a pressure reduction of the pressure system to 50% within 15 minutes. It consists of two consecutive valves, which are interconnected by a control system. In different operating states, both valves can be operated in supercritical states of flow. The system is designed for a sluggish pressure control.

In the publication DE 33 20 753 A1, a perforated plate is used for balancing the speed distribution in a flow channel. Multiple parallel flow holes evenly or rotation-symmetrically arranged are either formed as single or multi-stage shock diffusers or they are embodied with continuous expansion of the flow cross-sections.

FR 2 143 866 A1 relates to a throttling device for a pipeline having multiple parallel nozzles arranged in a throttle plate, which at rated flow cause a minimal pressure loss and limit the flow when the pressure drop through the throttles approaches the critical pressure. Preferably, the nozzles have a Venturi characteristic with converging inlet zones and diverging outlet zones. They are preferably inserted into holes of the throttle plate and are connected to the same by circumferential weld seams.

A similar construction is used in FR 2 710 392 A1. Here, a single cavitation Venturi nozzle in a pipeline is replaced with an arrangement of multiple smaller Venturi nozzles which have a natural frequency outside the operating range.

For testing such protective installations, a multiplicity of test devices, so-called shock tubes are known. In these mostly elongated test devices, an explosive gas is ignited and the effects on a test body are examined.

Tests with a shock tube of Langdon, 2008, https://www.researchgate.net/publication/267683072 Perforated Plates as Passive Mitigation System showed that two perforated plates arranged one behind the other exhibited a substantially more significant pressure mitigation than a perforated plate alone.

The protective installations against explosion pressures known from the prior art work with different mechanisms of action. Flame barriers, which extract heat energy from the hot gas, have a certain minimum length irrespective of their type of construction. The function components are only conditionally designed for pressure loads and require additional supports.

Porous media with sub-critical flow, such as for example, perforated plates are basically suitable for a mitigation of explosion pressures. However, the efficiency is limited and the advance calculation of the pressure mitigation is still subject to major uncertainties. Investigations are primarily available for use in pipelines. The realisations gained from this can only be conditionally applied to a large-area use closely before a tube base of a tube bundle reactor. Such tube bases can have diameters of up to 9 m and more.

Porous particulate media require a very large volume but have only a limited effect and require an expensive charging and discharging procedure.

Some flat devices have no explosion protection effects and merely serve for distributing the process gas.

Nozzles with critical flow can limit the flow in a highly defined manner. They are employed primarily as safety devices with one or few parallel throttles in pipelines.

Tube bundle reactors with supply of a process gas component during normal operation in the critical state of flow bring about high costs in terms of compressors and during the operation. In addition, the mixing quality is limited.

In tube bundle reactors with several metres diameter it has to be taken into account that the tube base has a temperature corresponding to the heat transfer medium, which circulates in the shell space of the tube bundle. In many cases, this temperature is in the range of approximately 300° C. to 500° C. A limit wall, such as, with the generic tube bundle reactor by contrast has the temperature of the first gas passing through it, which usually is in the range between 100° C. and 200° C. The consequence is that the tube base and the limit wall radially expand differently during the operation. When the braces are rigidly attached to the radial edge there is the risk that these break through overloading. With this construction it has to be additionally taken into account that the limit wall, even during the normal operation, is operated in the critical state of flow and even in this operating state causes a major loss of pressure with the consequence of high investment and operating costs.

In large tube bundle reactors with shell diameters in the range from approximately 8 to 9 m, the volume of the gas inlet region rises disproportionately. When such tube bundle reactors are additionally operated with an explosive process gas and additionally at elevated initial pressures in the range of approximately 2 to 5 bar absolute, the maximum expected explosion pressure increases greatly corresponding to the pressure increase factors of the process gas. Through the greater wall thicknesses and structural reinforcement caused by this one approaches technical and economic limits. This is true all the more when the inlet temperatures of the process gas are to be increased to approximately 150° C. to 200° C. This constitutes a particular challenge in the dimensioning of the tube bundle consisting of the reaction tubes and connected tube bases. Despite the variety of known devices for limiting the consequences of explosions in pipelines or other connected vessels, none of these is suitable to protect the reaction tube bundle of a tube bundle reactor within technically and economically acceptable limits during explosion pressure waves in the gas inlet region when explosive process gases with pressures of more than 2 bar absolute, and with high operating temperatures are used in the operation.

### SUMMARY OF THE INVENTION

Now, aspects of the invention are to remedy this. It is therefore based on improving a generic tube bundle reactor so that its reaction tube bundle can be reliably and cost-effectively protected against explosion pressure waves in the gas inlet region of the tube bundle reactor, even at high operating pressures and temperatures, and thereby impairs the normal operation as little as possible.

Through the measures according to one aspect of the invention, reaction tube bundles can also be protected during explosions in the gas inlet region in a functionally safe manner and with relatively low costs, even at high operating pressures and temperatures.

One aspect of the present invention utilises the physical effect that when a compressible fluid flows through a throttle upon the increase of the primary pressure under certain conditions a critical pressure ratio occurs. With this state of flow, the mass flow is merely linearly dependent on the primary pressure and no longer additionally on the outflow function. As a result, there is an automatic additional throttling. The mass flows for the corresponding sub-critical and critical state of flow are calculated, for example, according to the AD2000 information sheet A1.

One aspect of the invention is based on the realisation that upon arrangement of multiple such throttles on a throttle plate these throttles, upstream of a tube base generate a low pressure loss during the normal operation, but-when in the event of an upstream explosion the pressure loss increases greatly-throttle the outflow through the throttle plate, from the reaching of the critical pressure ratio, to a critical mass flow. The throttle plate designed according to one aspect of the invention, thus, results in two effects. On the one hand, a gas flow flows out through the throttle plate during the explosion. The pressure relief connected with this results, in the gas inlet space, in a maximum explosion pressure which is lower than the maximum static explosion pressure and which within the scope of this invention is referred to as “first reduced explosion pressure”. The maximum static explosion pressure forms when an explosive gas mixture in an enclosed vessel is caused to explode by ignition. This value can be calculated on the basis of the gas composition at a certain pressure and a certain temperature. Experimental determination methods are stated, for example, in DIN EN 15967:2022-03.

The second decisive effect consists in that through the throttled inflow to the intermediate space between throttle plate and opposite gas-inlet-side tube bottom a pressure increase is greatly dampened there. During the normal operation, the same gas rate flows through the throttle plate to the intermediate space as flows out into the reaction tubes. During an explosion in the gas inlet space, the inflow on the part of the throttle plate in the intermediate space increases to a greater degree than the outflow into the reaction tubes, so that in the intermediate space the pressure increases. In an unfavourable case, the flame front can strike through into the intermediate space and ignite the pre-compressed explosive gas situated there so that the explosion continues there. During the explosion, a gas flow flows out through the reaction tubes also in this case. The inflow from the throttle plate is obtained from the magnitude of the pressures that happen to be present on both sides of the throttle plate, wherein in the event of an explosion the mass flow is limited to the critical mass flow. Under certain conditions even a temporary backflow from the throttle plate into the gas inlet space is conceivable. In any case, the maximum explosion pressure in the intermediate space is also lower than the maximum static explosion pressure because of the resulting gas outflow. This resulting lower explosion pressure in the intermediate space is also referred to as “second reduced explosion pressure” within the scope of this invention. This second reduced explosion pressure is the lower, the smaller the volume of the intermediate space is. The influence will actually be very minor since the outflow area to the reaction tubes relative to the intermediate space volume is very large. Apart from this, the explosion striking through into the intermediate space will probably take place rather punctiformly and not suddenly over the entire area. Because of the disc-shaped volume, the explosion pressure will develop at a point and directly relieved through gas outflow into the opposite reaction tubes while other regions of the intermediate space have not been affected by the explosion wave.

For this it is sufficient to design all apparatus parts subjected to the first reduced explosion pressure, i.e. the apparatus parts of the gas inlet space, for this reduced explosion pressure in terms of strength and design all apparatus parts subjected to the second reduced explosion pressure, i.e. the apparatus parts of the intermediate space, to the second reduced explosion pressure of the same in terms of strength.

Because of the fact that according to one aspect of the invention the throttle openings have a narrowest flow cross-section and are arranged in a number with which in the event of an explosion at a pressure in the gas inlet space, which is maximally as high as the maximum static explosion pressure of the process gas and higher than the operating pressure during the normal operation and a pressure materialising in these conditions in the intermediate space between throttle plate and gas-inlet-side tube base a critical pressure ratio arises on the throttle plate so that as a result of the gas inflow from the throttle plate and of the gas outflow to the reactor tubes a predetermined pressure in the intermediate space between throttle plate and tube base is not exceeded, it is possible for the intermediate space to predetermine a desired second reduced explosion pressure at which the design of the apparatus parts of the intermediate space in terms of strength, such as, flange connection or tube base results in desired orders of magnitude. For example, the thickness of the gas-inlet-side tube base required for protecting the reaction tube bundle can be kept relatively small or the efficiency of the tube bundle reactor be increased, for example, larger number of reaction tubes (i.e. larger diameter of the reaction tube bundle, for example, 8 m and more) or higher explosion pressures.

Depending on the predetermined second reduced explosion pressure for the intermediate space and the critical pressure ratio to the gas pressure in the gas inlet space resulting from the design of the narrowest flow cross-section of the throttle openings according to the invention, a first reduced explosion pressure materialises there in the event of an explosion, on which the design of the apparatus parts of the gas inlet space in terms of strength is based.

Thus, with suitable dimensioning of the narrowest flow cross-section of the throttle openings the division of the reduced explosion pressures over the spaces upstream and downstream of the throttle plate can be adjusted in a defined manner. The reduced explosion pressure in the gas inlet space and in the intermediate space mutually influence one another. When the throttle resistance is increased, the second reduced explosion pressure in the intermediate space falls, while the first reduced explosion pressure in the gas inlet space rises and vice versa. In this way it can be achieved, for example, that support braces within the shell space of the tube bundle can be omitted while corresponding reinforcement measures in the gas inlet space can be achieved with less expenditure.

In particular, by varying the narrowest flow cross-section (i.e. of the critical pressure ratio) and of the second reduced explosion pressure, the distribution of the reduced explosion pressures over the gas inlet space and the intermediate space can be iteratively varied in order to optimise the design of the gas spaces in terms of strength based on this and minimise the overall manufacturing costs of the tube bundle reactor.

The narrowest flow cross-section and the number of the throttle openings are designed so that during the normal operation a sub-critical pressure ratio on the throttle plate between intermediate space and gas inlet space materialises. With a throttle plate designed according to one aspect of the invention, the pressure loss during normal operation is preferably in the range of 0.05 bar to 0.5 bar and particularly preferably in the range from 0.1 bar to 0.3 bar. Thus, it is small compared with the other flow resistances of the overall system; the main pressure loss through the catalyst bed alone is in the range from 0.4 bar to 1.0 bar.

In addition, a very good flow distribution over the tube base area materialises because of the design of the throttle plate according to one aspect of the invention; each reaction tube practically receives the same gas volumetric flow rate.

Apart from this, the entire gas inlet space is available as mixing section so that pending the entry of the processed gas into the reaction tubes a homogeneous mixture is achieved.

Usually, the gas inlet line is terminated at its upstream end by the compressor, which delivers the main part of the processed gas through the reactor system. For protecting upstream plant components, installations such as flame barriers, porous media or pressure shock cones are often provided. These are preferably arranged closely to the place from which an explosive gas is present, i.e. generally closely upstream of a mixer in which the process gas components are intermixed. Such installations are often able to extinguish a flame front. However, they are only capable of mitigating or stopping an explosion pressure wave to a limited degree. This applies all the more the greater the cross-section of the gas inlet line and the higher the operating pressure are.

Preferentially, the gas inlet space comprises an upstream limit, which particularly preferably is a non-return valve arrangement. The same is advantageously resistant to a downstream explosion pressure wave and able to mitigate the same at least so that upstream plant components are protected from damage.

In an advantageous aspect of the invention, the gas inlet hood, for minimising its gas-conducting volume, comprises at least one displacer or a correspondingly configured contour. By volume minimising, the dwell time and, thus, the risk of self-ignition is reduced. However, should an explosion occur, nevertheless, the portion of the gas flowing out through the throttle plate during this explosion increases, as a result of which the maximum explosion pressure falls.

Preferentially, the displacer or the wall of the gas inlet hood is connected to the throttle plate by anchors. The displacer is clamped at least to the throttle plate by anchors in order to avoid large wall thicknesses resulting from plate bending. The pressure force present in the space between displacer and throttle plate is, thus, largely absorbed by these anchors and no longer by the throttle plate and the displacer alone. The last mentioned parts can, thus, be embodied significantly thinner or make possible embodying larger tube bundle reactors. Advantageously, the anchors in turn support themselves against the gas inlet hood. The type of the anchors is not particularly restricted. A particularly small volume is taken up by round bars with circular cross-section, but other cross-sectional shapes such as, for example, ellipses or rectangles as well as corresponding hollow profiles are also possible. Furthermore, cylinders concentric to the reactor axis with corresponding gas passages are also possible. Since all these parts substantially have the same temperature, there is no additional loading through different thermal expansions.

By contrast, the intermediate space is free of anchors so that gas-inlet-side tube bases and throttle plate can radially expand differently without mutual interference.

In a favourable further development of the invention, the gas inlet hood comprises a gas inlet connector for connecting the gas inlet line and the gas inlet connector is extended up to the throttle plate and connected to the throttle plate. In this way, the thickness of the throttle plate can be even further reduced since the gas inlet connector serves as a further pressure-relieving anchor. The gas inlet connector is perforated in the region between displacer and throttle plate in order to make it possible for gas to pass through.

Preferably, the gas inlet hood is detachably connected to the gas-inlet-side tube base by a flange connection. This makes possible a free movement of the two parts relative to one another so that stresses from possible temperature differentials between these do not occur. It can be advantageously complemented by an anti-rotation device according to DE 20 2020 103 617 U1. During the operation of the tube bundle reactor, the process gas flows through the gas inlet line, the gas inlet hood and in the intermediate space between throttle plate and gas-inlet-side tube base with a temperature generally in the range from 100° C. to 300° C. All parts in contact with the process gas are nearly at gas temperature. When the process on the heat transfer medium side is driven at higher temperatures in the range from 300° C. up to 600° C., all apparatus parts in contact with the heat transfer medium are also at the temperature of the heat transfer medium. During the operation, the apparatus parts of the different temperature zones expand to different degrees. In the case of larger reactor diameters, for example, in the range between 6 and 8 m, the expansion differences between the gas inlet hood and the gas-inlet-side tube base become so great during the operation that in the case of simple welded constructions at the transition of the two temperature zones, material overloads can occur.

Since because of the throttle plate the pressure in the intermediate space directly before the tube base is significantly reduced in the event of an explosion, it is not only possible to design the gas-inlet-side tube base thinner, but the flange can also become significantly smaller.

Preferably, the flange connection is sealed with a weld lip seal. An embodiment according to DE 10 2020 127 779 B3 is advantageous. Through the low height of such a weld lip seal a particularly small volume of the intermediate space is obtained.

In a favourable further aspect of the invention, the throttle plate is arranged parallel to the gas-inlet-side tube base and the distance between throttle plate and tube base is in the range from 5 mm to 500 mm, preferably in the range from 10 mm to 200 mm and particularly preferably in the range from 20 mm to 70 mm. It cannot be ruled out that an explosion in the gas inlet space moves through the throttles in the throttle plate and ignites the explosive process gas in the intermediate space between throttle plate and gas-inlet-side tube base. Preferably, the intermediate space between throttle plate and tube base is embodied with reduced volume in order to keep the pressure wave during a possible explosion as small as possible there. The smaller this intermediate space is, the faster does the gas propagated with the explosion flows out into the reaction tubes so that the maximum explosion pressure is significantly dampened. The gas flowing out into the reaction tubes is rapidly cooled there so that the flame front is extinguished.

In an advantageous manner, the throttles of the throttle plate are arranged in a region which is substantially congruent with the tubed region of the gas-inlet-side tube base. This results in short flow paths. The throttles are preferably embodied identically.

Further, the number of the throttles in the region of the throttle plate, which is substantially congruent with the tubed region of the tube base, the number of the throttles is preferably smaller than the number of the reaction tubes. Thus, the production expenditure is reduced. The throttle openings are dimensioned accordingly. Fewer throttles require larger throttle openings, in the case of which the manufacturing tolerances can be more easily maintained and the risk of clogging through contaminations in the process gas is reduced.

It is advantageous when the ratio of the number of the throttles to the number of the reaction tubes is in the range from 1:6 to 1:1.5. The ratio is preferably in the range between 1:4 and 1:2 and is particularly preferably at 1:3. Through a smaller number of throttles, the manufacturing expenditure is reduced and larger throttle openings are obtained, the manufacturing tolerances of which are more easily maintained and in the case of which the service life of the processing tools is longer.

The axes of throttles and reaction tubes are preferably offset relative to one another, i.e. the gas jet of a throttle does not directly lead into a reaction tube, but strikes a region next to it, from where the gas is distributed over the adjacent reaction tubes. Thus, as even as possible a distribution of the process gases from the throttles to the reaction tubes is ensured.

Further preferably, the axes of throttles and reaction tubes are offset relative to one another in the operating state, i.e. taking into account different axially normal length expansions of throttle plate and tube base. These axially normal length expansions of throttle plate and tube base are determined dependent on the materials used and the operating temperatures expected and used as basis for determining the tube pattern or the throttle pattern. For the manufacture, the throttle pattern is subsequently used at room temperature or at a temperature at which the throttle pattern is produced.

Advantageously, the throttles and reaction tubes are each arranged in uniform patterns each with constant divisions. This results in a uniform distribution of the process gas from the throttles to the reaction tubes.

In a favourable configuration of one aspect of the invention, further throttles are provided in the throttle plate in regions which are situated opposite tube-free regions of the tube base, wherein the number of throttles per area (distribution density) in these regions amounts to between 2% and 20% of the distribution density of the throttles in the regions, which are situated opposite the tubed regions of the tube base. Besides the tubed regions of the tube base, there are also tube-free regions. In order to avoid stagnant gas in the intermediate space between the throttle plate and the tube base in these regions, which can ignite through self-ignition, these regions are advantageously flowed through as well.

The embodiment of the throttle bores is not particularly restricted. Simple bores with a diameter up to including flow-optimised throttles with Venturi or Laval characteristic are possible. In the manufacture of a throttle plate for a tube bundle reactor, manufacturing-based aspects have to be taken into account in particular. In the case of a tube bundle reactor with multiple tens of thousands of tubes a corresponding throttle plate has a number of throttles in the order of magnitude of multiple thousands of these. It has to be taken into account, furthermore, that the throttle plate has a thickness in the order of magnitude of approximately 20 to 60 mm. A continuous throttle opening in this depth and with this number is technically and economically unfavourable.

Preferably, the throttles are embodied as blind hole from the downstream side and have a centric throttle opening between blind hole and upstream side.

Such a throttle opening can be produced with relatively little expenditure and can be easily automated. In a first step, blind holes are produced on the downstream side of the throttle plate according to a drill pattern corresponding to criteria described above. According to strength-related criteria, the diameter should be small in order to mitigate the throttle plate as little as possible. From a flow point of view the diameter should be as large as possible in order to give the gas flow, having flowed through the throttle opening, sufficient running length for forming an undisturbed flow profile. According to this aspect, the open end of the blind hole should also be considered as diffuser opening. As a compromise, a bore diameter in the range between 8 and 15 mm is preferably used.

The blind hole ends closely before the end of the throttle plate, so that a minimal distance in the range between 2 and 10 mm remains. The diameter of the throttle bore is obtained as result of the considerations described above and generally is in a range between 2 and 5 mm. Although the side of the throttle plate from which the throttle bore is produced is basically the same, drilling is preferably performed from the side of the blind hole since through the conical end of the blind hole the throttle bore is necessarily centred on the axis of the blind hole bore. Apart from this, the blind hole can be used for a guiding device. On the upstream side, the throttle bore is practically at least deburred or possibly even chamfered. For calculating the flow through a throttle bore, the associated outflow figure is required. Although there are table values, but these can only approximate the behaviour of the special geometry of the throttle bore of the respective application case.

To increase the calculation accuracy, a sample body with the dimensions of the described throttle is preferably produced and the flow characteristics experimentally determined by this.

With the method described above, a throttle plate with given specification using adequate automation means can be produced relatively quickly and economically. However, each bore has only a limited service life and the drilling tolerances can easily leave the permissible ranges when this is not frequently enough checked in between or when there are material defects. Especially when there are defects in the throttle bores and these may have become too large in diameter it is difficult to rectify this defect.

In an advantageous aspect of the invention, the throttles can therefore be each embodied as insert part, which is inserted into a bore of the throttle plate.

In this procedure, the region with a large diameter of the throttle opening is drilled through completely. Compared with the production of a blind hole, this operation requires significantly less effort. The throttle as insert part can be produced as mass-produced part by specialised companies with high degree of automation and high dimensional accuracy. Here, the degree of detailing of the throttle opening as described above can be established as desired. A further advantage of the insert part solution consists in that defective throttles can be subsequently replaced. Furthermore, experimental throttle curves can be more easily determined with this embodiment and a comparison with the analytical calculation be carried out which increases the reliability of the overall construction.

For fixing the insert part in the through bore, several methods are possible. A simple solution consists in embodying the insert part with an outer diameter that is slightly smaller than the through bore and equip the end with a collar which is welded to the throttle plate. Furthermore, a screw-in solution is also possible.

With the abovementioned aspects of throttle openings, defined flow resistances can be achieved in a simple manner. Further, such configurations of the throttle openings result in a relatively large narrowest flow cross-section, as a result of which the safety against clogging is increased.

It is advantageous when a tube bundle reactor according to the invention comprises at least one additional throttle plate which is arranged at a given distance parallel to the first throttle plate and is likewise tightly connected to the gas inlet hood at its circumferential edge. Thus, an even more significant mitigation of the explosion pressure is achieved.

The tube bundle reactor is particularly suited for a normal operating pressure in the gas inlet space in the range from 2.0 to 7.0 bar absolute.

The invention also includes the use of a tube bundle reactor according to any one of the preceding claims for oxidation processes, in particular for oxidative dehydrogenation reactions, the oxidation of propene to acrolein, the oxidation of butane to maleic anhydride and the oxidation of naphthalene and/or o-xylol, if applicable in mixing mode, into phthalic anhydride.

### BRIEF DESCRIPTION OF THE DRAWINGS

As an example, the invention is described in more detail in the following by way of the drawings. There it shows:

FIG. 1: a vertical section through a schematic representation of an embodiment of a tube bundle reactor;

FIG. 2A: an enlarged representation of the flange region II of the tube bundle reactor shown in FIG. 1;

FIG. 2B: a variant of the enlargement shown in FIG. 2a;

FIG. 3A: in cross-section the part III of the throttle plate shown in FIG. 2a with a throttle configured as a throttle opening drilled directly into the throttle plate;

FIG. 3B: a representation similar to FIG. 3a with a throttle configured as inserted and welded insert part;

FIG. 3C: a representation similar to FIG. 3a with a throttle configured as a screw-in insert part;

FIG. 3D: a representation similar to FIG. 3a with a throttle configured as an inserted insert part with Venturi characteristic of the throttle opening;

FIG. 4: in a plan view in the direction of the arrow IV in FIGS. 2a and 2b an example of a distribution pattern of throttles and reaction tubes.

### DETAILED DESCRIPTION OF THE PRESENTLY PREFERRED EMBODIMENTS

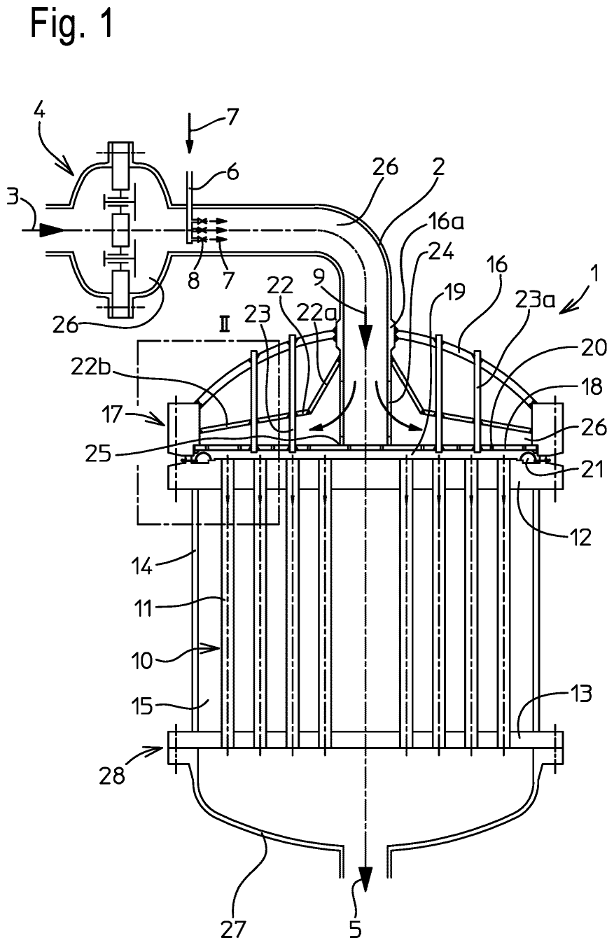

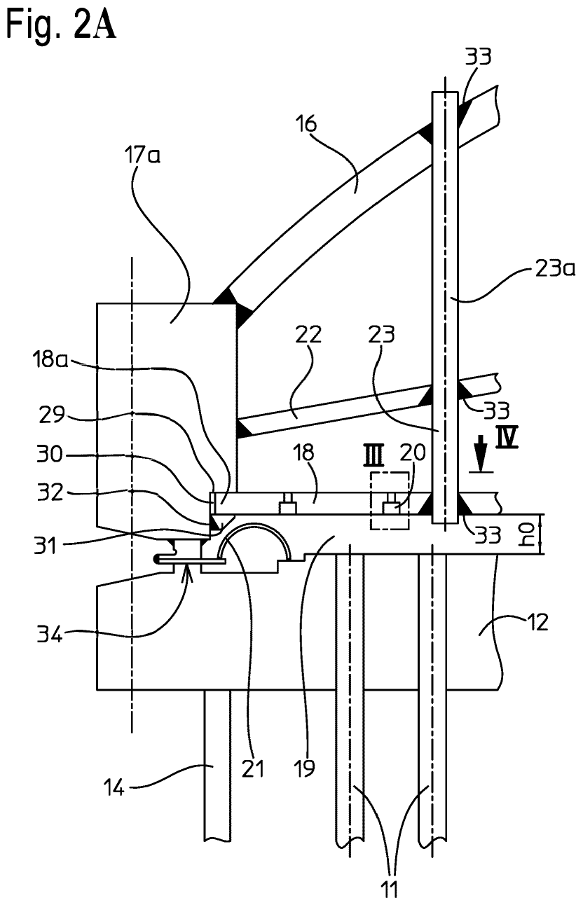

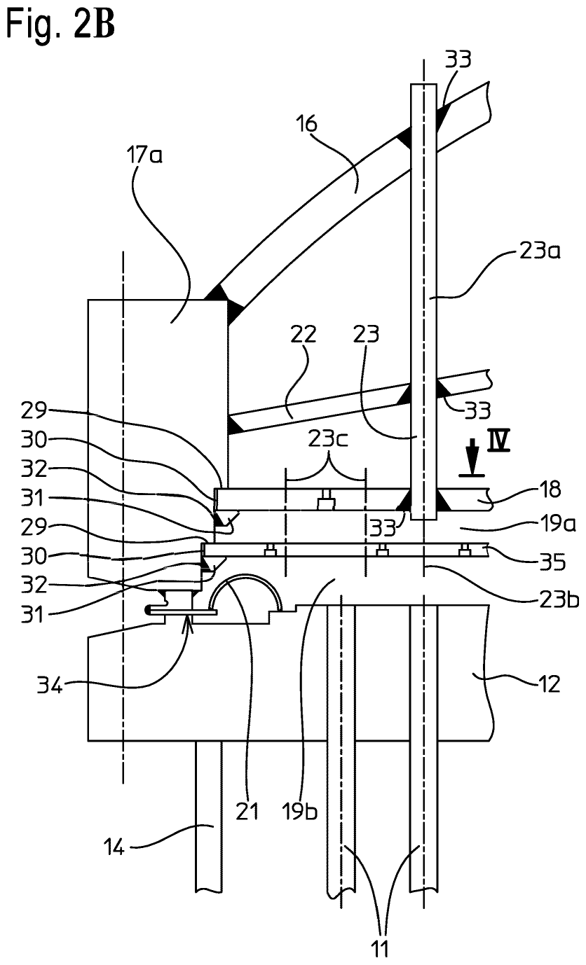

FIG. 1 and FIG. 2a with an enlarged region II from FIG. 1, show an exemplary embodiment of a tube bundle reactor 1 according to the invention with connected gas inlet line 2. In the shown exemplary embodiment, a first non-explosive process gas component 3 flows through a known non-return valve device 4, as described in WO 2004/067164 A1 or in DE 20 2005 009 825 U1, into the gas inlet line 2. The non-return valve arrangement 4, therefore, forms the upstream limit of the gas inlet line 2 with respect to the event of an explosion. The first process component 3 is conditioned with preceding means in terms of its composition and has a predetermined temperature and a predetermined pressure. It includes, for example, a gas containing oxygen and where appropriate inert and/or non-converted components of the product gas 5. Downstream following the non-return valve arrangement 4 a dosing apparatus 6 for dosing a second process component 7 is arranged. This second process gas component 7 contains at least one combustible starting material, which is provided as educt for the given process. It can be gaseous or liquid and is added, for example, into the main gas flow using nozzles 8. The second process gas component 7 is either mixed with the first process gas component 3 into a homogeneous process gas 9 either already by the dosing apparatus 8 or with mixing devices in the gas inlet line 2, not shown here, or with combinations thereof. This gas mixture now forms an explosive process gas 9. This process gas 9 can also be produced by many different methods which are not described here.

The tube bundle reactor 1 comprises a reactor main part, which is formed from a bundle 10 of catalyst-containing reaction tubes 11. The reaction tubes 11 are attached to an upper gas-inlet-side tube base 12 and a lower gas outlet-side tube base 13. The tube bundle 10 is surrounded by a cylindrical reactor shell 14, which is likewise connected to the upper and the lower tube base 12, 13. By way of supply and discharge lines, which are not shown here, a heat transfer medium is fed into and discharged from the shell space 15, with which the reaction heat generated in the reaction tubes 11 is dissipated. The temperature control system necessary for this, comprising, for example, circulating pumps, coolers, control valves, baffle plates in the shell space 15 of the tube bundle reactor 1 and other control devices are known to the person skilled in the art. The different parts of the tube bundle reactor 1 are interconnected with connecting techniques known for this purpose so that this will not be discussed further here.

The upper gas-inlet-side tube base 12 is spanned by a gas inlet hood 16. The same is releasably connected to the tube base 12 with a flange connection 17. Here, the flange 17a is embodied similar to a block flange. The process gas 9 is conducted through the gas inlet line 2 into the gas inlet hood 16. Preferably, the gas inlet line 2 between non-return valve device 4 and gas inlet hood 16 is kept as short as possible in order to keep the effects of an explosion as low as possible. For protecting the upper tube base 12 from an explosion pressure wave, a throttle plate 18 is arranged with short distance h0 parallel to the same so that between the upper tube base 12 and the throttle plate 18 an intermediate space 19 is formed. At its outer edge the throttle plate 18 is connected to the flange 17a of the gas inlet hood 16 and contains a multiplicity of throttles 20 through which the process gas 9 flows before it enters the reaction tubes 11. During the operation, the upper tube base 12 and the gas inlet hood 16 can expand to a different degree because of different temperatures. For this reason, the flange connection is sealed with a weld lip seal 21. Thus, the tube base 12 and the throttle plate 18 can move relative to one another without stress and the intermediate space 19 is additionally hermetically sealed against the process gas 9 leaking into the environment.

The gas-conducting volume of the gas inlet hood 16 is minimised by a displacer 22, which in the shown exemplary embodiment is formed out of two conical sections 22a, 22b. The pressure forces of an explosion both on the throttle plate 18 and also on the displacer 22 are absorbed by different braces, as a result of which these parts can be embodied relatively thin. A first kind of the bracing consists in anchors 23 between the throttle plate 18 and the displacer 22. These anchors 23 can be embodied with different cross-sections. Preferred, however, are round bars since this profile obstructs the flow the least and can be easily procured. A second kind of the bracing is formed in that the gas inlet line 2 is passed through the gas inlet hood 16 and continued as far as to the throttle plate 18. In this region, the gas inlet line 2 is equipped with windows 24 which make possible a flow of the process gas 9 along the throttle plate 18 which is radially directed outwardly. The windows 24 can also be replaced with any porous media or perforated plates with constant or locally distinct porosity. In a modification, the gas inlet hood 16 can comprise a gas inlet connector 16 a for connecting the gas inlet line 2 and the gas inlet connector 16 a and the gas inlet connector 16a be extended as far as to the throttle plate 18 and connected to the throttle plate 18. Preferably, the anchors 23 are welded to the throttle plate 18 and to the displacer 22. Likewise, the lower end 25 of the gas inlet line 2 is preferably welded to the throttle plate 18. However, it is also possible to embody the gas inlet line 2 in the region between throttle plate 18 and displacer 22 by screw connections of segmented sections. This is advantageous for inspection and maintenance work. With the shown construction, the pressure space within the tube bundle reactor 1 is formed solely by the throttle plate 18, the displacer 22 and the flange 17a. Therefore, the gas inlet hood 16 would not be required. However, for the event that an explosion occurs in the intermediate space 19 between tube base 12 and throttle plate 18, the throttle plate 18 and the displacer 22 should be considered as unit which are subjected from the outside-here, from the intermediate space 19—by a pressure force. This pressure force is preferably absorbed in that the aforementioned anchors 23 between throttle plate 18 and displacer 22 are run with extensions 23a as far as to the gas inlet hood 16. For manufacturing reasons, these are advantageously passed through the gas inlet hood 16 and welded to the same from the outside.

The space between the upstream limit of the gas inlet line 2 flowed through by gas, which in the shown exemplary embodiment is formed by the non-return valve device 4, and the throttle plate 18 is referred to as gas inlet space 26 within the scope of this invention.

During the operation, the gas-inlet-side tube base 12 is exposed to different heat influences. On the gas-inlet-side, process gas 9 flows with gas inlet temperature. The process gas 9 flows through the reaction tubes 11, which on the gas-inlet-side are connected to the upper tube base 12. On the heat transfer medium side, the upper tube base 12 is in contact with the heat transfer medium. Through the greatly different temperatures on both sides of the upper tube base 12, the upper tube base 12 on the side of the heat transfer medium expands to a greater degree than on the side of the process gas inlet. As a consequence, the upper tube base 12 has a tendency of assuming a key-like shape as a result of which the tightness of the flange 17a is no longer guaranteed. This is a further reason for employing the weld lip seal 21.

At its lower end, the tube bundle reactor 1 is terminated by a gas outlet hood 27, which in the known manner is connected to the reactor main part. This can take place through the flange connection 28 as shown in FIG. 1 or through a welded connection. The tube bundle reactor 1 in the shown exemplary embodiment is flowed through by process gas 9 from the top down. However, a flow from the bottom up is also possible. In this case, the tube bundle reactor 1 shown in FIG. 1 is turned by 180° and the description of all reactor components is correspondingly adapted. It is to be understood that all parts subjected to an explosion pressure are designed to this load in terms of strength. Further advantageous details for the configuration of a tube bundle reactor with explosion pressure loads can be taken from WO 2004/067164 A1.

Should an explosion occur in the gas inlet space 26 a conservative assumption is that the explosive gas in the gas inlet space 26 is ignited in a place where an explosive gas is present for the first time. Generally, this is in the region of the dosing apparatus 6 for the second process gas component 7. A propagation of the explosion pressure wave upstream will only take place to a limited degree since the gas velocity in such applications is usually in the range between 20 m/s and 30 m/s and a propagation of the flame front upstream is rather improbable. Nevertheless, the pressure wave will also spread upstream. By way of the non-return valve device 4, this explosion pressure wave directed upstream can be stopped or at least mitigated so far that in upstream plant components no damage occurs. Following the start of an explosion, the flame front moves downstream with a propagation speed corresponding to the properties of the explosive gas. The pressure in this gas inlet space 26 is approximately the same in any location.

While the throttles 20 in the throttle plate 18 are flowed through in the sub-critical state of flow during normal operation, these are flowed through in the critical state of flow in the event of an explosion of the gas inlet mixture or of the process gas 9 in the gas inlet space 26 and the gas flow is limited to the critical mass flow. The throttles 20 have to be dimensioned so that in the event of an explosion a first reduced explosion pressure materialises in the gas inlet space 26 taking into account a gas outflow from the gas inlet space 26 via the throttles 20 into the intermediate space 19 and that in the intermediate space 19, taking into account the gas inflow from the gas inlet space 26 and a gas outflow into the reaction tubes 11, a second reduced explosion pressure materialises, wherein the first and the second reduced explosion pressure are the basis for determining a first and a second calculation pressure respectively. According to the invention, the throttles 20 are dimensioned so that a predetermined pressure in the intermediate space 19 is not exceeded. With this procedure, the expenditure for producing the apparatus is minimised.

In FIG. 2a, the flange region II shown in FIG. 1 on the gas inlet side is shown enlarged. The throttle plate 18 is not welded to the flange 17a. It 18 lies against a shoulder 29 of the flange 17a with radial play 30 and is axially fixed by a locking ring 31, which is connected with a weld seam 32 to the flange 17a. In this way, the throttle plate 18 is axially fixed but freely movable in the radial direction. Thus, material loads at the contact point are omitted when the flange 17a and throttle plate 18 should expand differently as a consequence of different temperatures. Apart from this, intrinsic stresses in the throttle plate 18 are avoided which could develop during the welding operation. This arrangement is also possible because a complete seal of the throttle plate 18 against the flange 17a is not necessary. A minor leakage is even advantageous since stagnant gas in the region of the weld lip seal 21 is thereby avoided.

The position of the weld seams 33 at the anchors 23 to the displacer 22, the throttle plate 18 and to the gas inlet hood 16 is fixed in the shown exemplary embodiment as is optimal from a welding point of view. In a variant, which is not shown here, it is possible, furthermore, to connect the end of the anchor 23 to the throttle plate 18 so that the throttle plate 18 is similarly movable as in its edge region 18a. For this purpose, perforated disc-shaped stops on both sides of the throttle plate 18 are welded to the anchor 23. This embodiment is advantageous, for example, in particular when the throttle plate 18 is embodied with a material which has a different thermal expansion coefficient than the adjoining components or when welding the two materials to one another is difficult. The weld lip seal 21 is shown in detail here. It makes possible a radially free movement of the tube base 12 and the flange 17a relative to one another on a sliding plane 34. By using an embodiment according to patent DE 10 2020 127 779 B3, a small distance h0 and thus a small explosion volume is advantageously achieved.

FIG. 2b shows a variant of the throttle plate from FIG. 2a. There, a second throttle plate 35 is arranged downstream of the first throttle plate 18 parallel to and at a given distance to the same. By way of throttle plate 18 and throttle plate 35, a first intermediate space 19a is formed. Through throttle plate 18 and throttle plate 35 a first intermediate space 19a is formed. The configuration of this second throttle plate 35 takes place based on the principles regarding the throttle plate 18. Since the main load of the explosion pressure is already absorbed by the first throttle plate 18, the throttle plate 35 can be embodied correspondingly thinner. Like the first throttle plate 18, it can only absorb limited pressure differentials on its own because of the large reactor diameter and is therefore braced with the first throttle plate 18 by extensions 23b of the already present anchors 23. Additional connecting anchors 23c between the two throttle plates 18 and 35 can be additionally provided. In a preferred embodiment, these braces comprise releasable connections. The second throttle plate 35 is connected to the flange 17a in the radial direction in a manner similar to the first throttle plate 18. The throttle plate 35 forms a second intermediate space 19b with the tube base 12.

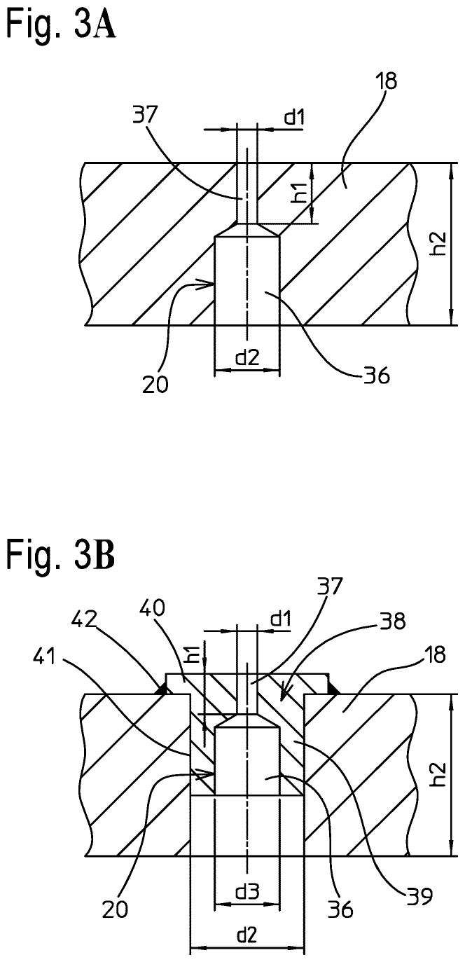

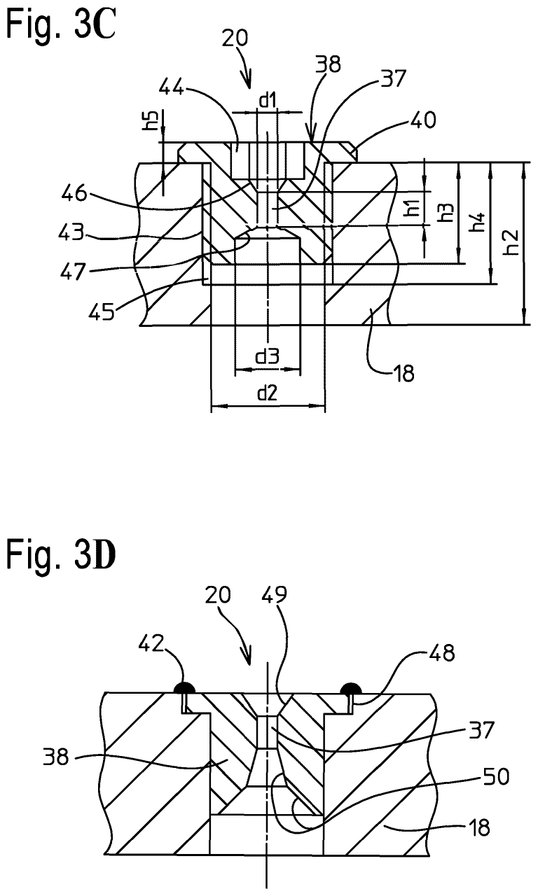

FIG. 3 shows a simple embodiment of a throttle 20. This is embodied as bore directly in the throttle plate 18, namely as blind hole 36 from the downstream side and as a centric throttle opening 37 between blind hole 36 and upstream side of the throttle plate 18. In a first step, the blind hole 36 with a diameter d2 is initially drilled into the throttle plate 18, which has a plate thickness h2, wherein a minimal distance h1 to the upstream side of the throttle plate 18 remains. In a second step, a throttle bore 37 with a diameter d1 is produced.

When there are greater requirements in terms of the dimensional stability of the throttle openings 20, the same can be produced as insert part as shown in FIG. 3b. This manufacturing method has the advantage that it can be produced as mass-produced part quickly and with high dimensional stability by specialised technical firms. The quality control of such a small part is substantially more reliable in a testing station than is possible with direct manufacture according to FIG. 3a. Manufacturing defects can also be more quickly detected here and countermeasures be taken. In the shown exemplary embodiment, the insert part 38 is formed out of a simple cylindrical part 39 with a collar 40 and comprises a blind hole 36 with a diameter d3 from the downstream side and a centric throttle opening 37 with a diameter d1 between blind hole 36 and upstream side of the insert part 38. It sits in a through-bore 41 in the throttle plate 18, wherein the collar 40 lies against the upstream side of the throttle plate 18 and the insert part 38 extends only over a part of the length of the through-bore 41. For installation, the through-hole 41 with a diameter d2 is initially drilled into the throttle plate 18. Then, the insert part 38 is inserted into the through-hole 41. Because of the collar 40, the insert part 38 cannot slip through and is unambiguously fixed in its position. The position is fixed through a weld seam which is so strong that it securely fixes the insert part 38 even in the event of a downstream explosion.

Through the two-stage expansion of initially the throttle diameter d1 to d3 and subsequently from d3 to d2, the pressure loss can be easily minimised. The fit at d2 between the bore 41 and the insert part 38 is determined as practical. The insert part 38 as well as variants thereof can also be inserted from the downstream side of the throttle plate 18. In this case, the throttle bores have to be suitably adapted. In FIG. 3c, a variant of the insert part 38 according to FIG. 3b is shown. The insert part 38 is embodied as releasable connection with screw thread 43. For screwing in, inner or outer contours can be provided on the upstream side of the insert parts 38, for example, an internal hexagon 44 or an external hexagon on the collar 40. Furthermore, this embodiment requires an internal thread 45 with a height h4 in the throttle plate 18 and an external thread with a height h3 on the insert part. With automatic machine production, this insert part 38 can also be produced economically. During the course of this, further advantageous features can be realised, such as for example, an inlet cone 46 before the throttle bore 37 and an outlet cone 47 after the throttle bore 37. The advantage is the simple installation and removal upon initial installation or replacement. The construction can be complemented by any other anti-rotation devices.

FIG. 3d represents a further variant of FIG. 3b. Here, the insert part 38 is inserted into a depression 48 so that it terminates flush with the upstream surface of the throttle plate 18. Furthermore, the pressure loss of this throttle 20 is reduced through a conical inlet section 49 and two downstream diffuser sections 50. The position is fixed by a weld seam 42.

Obviously, all bore characteristics, manufacturing methods and fixing methods shown here can be combined with one another as desired and complemented with other practical features.

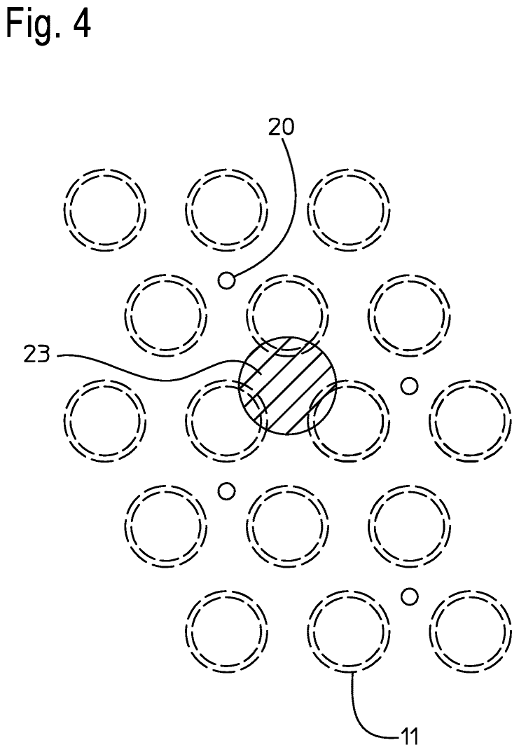

In FIG. 4, an exemplary embodiment of a distribution pattern of throttles 20 and reaction tubes 11 is shown. With this arrangement, an exemplary ratio of throttles 20 to reaction tubes 11 of 1:3 and an anchor 23 are shown.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1.-18. (Canceled)

19. A tube bundle reactor for carrying out catalytic gas phase reactions, comprising:
a bundle of catalyst-filled reaction tubes, which are flowed through by a process gas with a predetermined operating pressure and with a predetermined operating temperature, which during normal operation either has an explosive gas composition or a gas composition which during the normal operation is non-explosive and in a non-operational case, has an explosive gas composition;
a gas-inlet-side tube base, which is tightly connected to gas-inlet-side ends of the reaction tubes and through which the reaction tubes lead into a gas inlet hood,
wherein the gas inlet hood spans and is tightly connected to the gas-inlet-side tube base;
a gas inlet line, which leads into the gas inlet hood into which it conducts a gas;
a throttle plate, which upstream of the gas-inlet-side tube base, is arranged at a predetermined distance from the gas-inlet-side tube base and at its circumferential edge is connected to the gas inlet hood and thus forms an intermediate space between the throttle plate and the gas-inlet-side tube base and comprises a predetermined number of throttle openings,
wherein all parts of the tube bundle reactor and the gas inlet line directly or indirectly exposed to an explosion pressure are designed correspondingly resistant in terms of strength,
wherein
the gas inlet line is configured to introduce explosive process gas into the gas inlet hood and the upstream end of the gas inlet line is terminated by a limit, so that between the limit and the throttle plate a gas inlet space is formed, and
the throttle openings have a narrowest flow cross-section and are arranged in a number with which
during normal operation, a sub-critical pressure ratio on the throttle plate between intermediate space and gas inlet space materialises and
with which in event of an explosion upon a pressure in the gas inlet space which is maximally as high as a maximum static explosion pressure of the process gas and higher than the operating pressure during normal operation, and a pressure materialising in these conditions in the intermediate space between throttle plate and gas-inlet-side tube base a critical pressure ratio on the throttle plate materialises, so that
as result of gas inflow from the throttle plate and of gas outflow to the reaction tubes a predetermined pressure in the intermediate space between throttle plate and tube base is not exceeded.

20. The tube bundle reactor according to claim 19, wherein the gas inlet hood for minimising its gas-conducting volume comprises at least one displacer or a correspondingly configured contour.

21. The tube bundle reactor according to claim 20, wherein the at least one displacer or a wall of the gas inlet hood is connected to the throttle plate by anchors.

22. The tube bundle reactor according to claim 19, wherein the gas inlet hood comprises a gas inlet connector for connecting the gas inlet line and the gas inlet connector is extended as far as to the throttle plate and connected to the throttle plate.

23. The tube bundle reactor according to claim 19, wherein the gas inlet hood is releasably connected to the gas-inlet-side tube base by a flange connection.

24. The tube bundle reactor according to claim 23, wherein the flange connection is sealed with a weld lip seal.

25. The tube bundle reactor according to claim 19, wherein the throttle plate is arranged parallel to the gas-inlet-side tube base and the distance between throttle plate and tube base is in a range from at least one of:
5 mmm to 500 mm,
10 mm to 200 mm, and
20 mm to 70 mm.

26. The tube bundle reactor according to claim 19, wherein the throttles of the throttle plate are arranged in a region which is substantially congruent with the tubed region of the gas-inlet-side tube base.

27. The tube bundle reactor according to claim 26, wherein in the region of the throttle plate which is largely congruent with the tubed region of the tube base, a number of the throttles is smaller than a number of the reaction tubes.

28. The tube bundle reactor according to claim 27, wherein the ratio of the number of the throttles to the number of the reaction tubes is in a range from 1:6 to 1:1.5.

29. The tube bundle reactor according to claim 27, wherein axes of throttles and reaction tubes are offset relative to one another.

30. The tube bundle reactor according to claim 26, wherein the throttles and reaction tubes are each arranged in uniform patterns each with constant divisions.

31. The tube bundle reactor according to claim 26, wherein in the throttle plate in regions situated opposite tube-free regions of the tube base, further throttles are provided, wherein the number of the throttles per area (distribution density) in these regions is between 2% and 20% of the distribution density of the throttles in the regions which are situated opposite the tubed regions of the tube base.

32. The tube bundle reactor according to claim 19, wherein the throttles from a downstream side are embodied as blind hole and have a centric throttle opening between blind hole and upstream side.

33. The tube bundle reactor according to claim 19, wherein the throttles are each embodied as insert part, which is inserted into a bore of the throttle plate.

34. The tube bundle reactor according to claim 19, characterised by at least one additional throttle plate which is arranged at a predetermined distance parallel to a first throttle plate and likewise at its circumferential edge is connected to the gas inlet hood.

35. The tube bundle reactor according to claim 19, wherein in the gas inlet space during normal operation a pressure in a range from 2.0 to 7.0 bar absolute is present.

36. The tube bundle reactor according to claim 19 configured for an oxidation process configured as one of:
an oxidative dehydrogenation reaction;
an oxidation of propene to acrolein;
an oxidation of butane to maleic anhydride; and
an oxidation of naphthalene and/or o-xylol, if applicable in mixing mode, into phthalic anhydride.
